(19) United States
(12) Patent Application Publication
(10) Pub. No.: US 2011/0153029 A1
(43) Pub. Date: Jun. 23, 2011

(54) BIORESORBABLE AND FLEXIBLE MEMBRANES EXHIBITING ASYMMETRIC OSTEOCONDUCTIVE BEHAVIOR IN BOTH FACES

(76) Inventor: João Filipe Colardelle Mano, Guimaraes (PT)

(21) Appl. No.: 13/002,265

(22) PCT Filed: Jun. 29, 2009
(86) PCT No.: PCT/IB2009/052812

§ 371 Date: Feb. 7, 2011

(30) Foreign Application Priority Data
Jun. 30, 2008 (PT) 104119

(51) Int. Cl. A61F 2/28; A61L 33/00

(52) U.S. Cl. 623/23.61; 427/2.1

(57) ABSTRACT

The present invention concerns a process for the preparation of a flexible composite membrane, biocompatible and biodegradable, obtained from natural or synthetic materials, containing inorganic osteoconductive elements with a gradient of concentration along the thickness of the membrane or an asymmetric concentration of such elements in both sides of the membrane. The membrane may be obtained by gravitational deposition of the osteoconductive elements during solvent evaporation. The membrane is aimed to be used in vivo, in guided tissue regeneration, as a sealant for defects or structures for tissue engineering, or as coatings for biomaterials. The membrane exhibits one side featuring osteoconductive properties, that interacts favorably with mineralized tissues or other implanted bioactive materials, and the other side prevents the penetration or invasion of other tissues.

### DOMAIN OF THE INVENTION

The present invention concerns the preparation of a flexible composite membrane, biocompatible and bioresorbable, that contains inorganic osteoconductive elements with a gradient of concentration along the thickness or an asymmetric concentration of such elements in both sides. This invention may be applied to medicine or veterinarian medicine, for both regenerative therapies related to mineralized tissues or implantology.

### BACKGROUND OF THE INVENTION

Guided Tissue Regeneration (GTR) is a well established therapeutic tool that permits the reconstruction treatment of damaged tissues using a membrane that isolates the defect region from the invasion of other tissues, mainly connective tissue. Periodontal diseases are frequent in Humans and constitutes, together with dental caries, the principal cause of teeth loss in adults. The cicatrization of gum or bone tissue upon periodontal surgeries, such as teeth removal, results in a preferential formation of soft connective tissue rather than bone tissue. Bone resorption after teeth extraction is continuous in alveolar bone, making more difficult posterior implantations e also the recovery of the aesthetic gum contours.

GTR has been used in the restoration of periodontal tissues (periodontal ligament, alveolar bone, cementum and dentin) (H. L. Wang, J. Periodont. 76 (2005) 1601), maxillofacial defects or long bone defect. Bone defects are usually filled with autologous or allogeneic grafts or non-biological osteoconductive biomaterials. In this context bioactive biomaterials are recognized as systems that interact favorably with surrounded mineralized tissues, improving the osteointegration of the implanted device, inducing the formation of healthy tissue and avoiding the occurrence of necrosis.

In many situations, forces are generated from adjacent tissues, exerting pressure into the defects of mineralized tissues, also promoting the invasion of such soft tissues in the defect regions. The migration of these soft tissues could disturb or totally impede the osteogenesis on the defect or the surgical area.

Much effort has been done in developing or improving devices and methods that could facilitate the regeneration of tissues in defined regions, by minimizing the effect of the pressure inflicted by surrounded soft tissues or preventing the penetration of conjunctive and epithelial tissue. A field of fast expansion corresponds to the use of tissue engineering strategies for the regeneration of tissues and organs. Typical procedures involve the implantation of an hybrid system that combines a support material, usually a porous biodegradable structure or an hydrogel, and cells, often combined with soluble factors. In some cases, only the material component is implanted, and the regeneration may take place through the recruitment of cells from the surrounding tissues towards the biomaterial structure. It may be convenient the use of membranes to seal and isolate the anatomic region containing the tissue engineering device from other tissues.

Membranes based on poly(lactic acid) or poly(glycolic acid) have been proposed for GTR but, although exhibiting good biocompatibility, they don't exhibit osteoconductive properties (see, for example patent WO9324097). More advanced systems for GTR, such as the one referred in patent US2006149392, utilizes porous materials containing growth factors that also promote the development of bone or cartilage.

Composite systems containing poly(D,L-lactic acid) and Bioglass® (a bioactive glass), have been proposed for the regeneration of bone tissue (J. A. Roether et al., Biomaterials 23 (2002) 3871), including in the form of membranes (O. Tsigkou et al., J. Biomed. Mater. Res. 80A (2007) 837). Thin membranes of mineralized collagen have also been proposed in GTR applications, as refereed in U.S. Pat. No. 6,417,166. The used technology to process such composite membranes did not allow to obtain films with a gradient or an asymmetry of concentration of the inorganic component.

The biological environment felt by the two sides of the membranes is quite different for the applications abovementioned. There has been some effort to develop membranes for GTR exhibiting distinct properties in the two sides. Patent CN1586637 propose the preparation of flexible membranes with two layers, composed by collagen and hyaluronic acid, for GTR. In patent WO1999/019005 non-bioactive membranes produced by collagen multilayers were proposed for GTR, to be applied in cartilage and bone.

For specific orthopedic and implantology applications, it would be desirable to combine in the membranes both asymmetry and osteoconductive properties. In patent US2008044449 flexible membranes are described, exhibiting, at least in one of the faces, inorganic granulated material containing calcium, in order to promote bioactivity. The osteoconductive face is populated with proturbances presenting exposed regions of the granules. Composite membranes combining nanofibers of gelatin and hydroxyapatite were developed for GTR (Hae-Won Kim et al., Adv. Funct. Mater. 15 (2005) 1988); the work reefers the interest in developing systems with a compositional gradient of hydroxyapatite, suggesting that the stacking of multilayers could be a way to fabricate such films. S. Liao et al. (Biomaterials 26 (2005) 7564; Dental Mater. 23 (2007) 1120) proposed new composite compact membranes, characterized by different concentrations of hydroxyapatite nanoparticles in the two faces; in this case the membranes are composed by three layers that are stacked together. The strategies involving the production of membranes exhibiting asymmetric osteoconductivity that have been proposed before are based on the assembly of independent homogeneous composite membranes.

The present invention propose a biodegradable membrane fabricated as a single layer, using a single processing step, in which the gradient or the asymmetry in concentration of the bioactive component do not exhibit a discontinuity along the thickness of the membrane. Such possibility represents an advance in the current state-of-the-art as it decreases the complexity and the production time of the membranes, and prevents any occurrence of delamination that could take place in the multi-layer solutions.

### SUMMARY OF THE INVENTION

As an effort for the development of new materials and concepts for GTR, this invention proposes the production and use of a biodegradable membrane containing a bioactive component with a gradient or asymmetry of concentration along the thickness of the membrane without the need of stacking different layers, prepared, for example, by a single step. Such characteristic prevent possible delamination problems and decrease the complexity and production time of these membranes.

The proposed method includes: (i) the process to obtain a polymeric membrane for in-vivo applications exhibiting a gradient or asymmetry in the concentration of osteoconductive elements along the thickness of the membrane; (ii) the use of such a bioresorbable membrane with asymmetric bioactivity in applications related to guided tissue regeneration, regenerative medicine or reconstructive medicine, implantology or as a coating for biomaterials in orthopedic or odontological applications.

### GENERAL DESCRIPTION

The present invention concerns the preparation of a composite flexible membrane, biocompatible and biodegradable, obtained from natural-based or synthetic materials, that contains inorganic osteoconductive elements featuring a gradient or asymmetry in concentrations along the thickness of the membrane. Such inorganic osteoconductive elements are hereby designed by particles.

The membrane is constituted by, at least, one polymeric material and, at least, one osteoconductive material.

The polymeric material is selected among the family of the biodegradable synthetic polymers (for example, polyesters such as the derivative of poly(lactic acid) or poly(glycolic acid)), or macromolecules from natural origin, including polysaccharides (for example derivatives of chitin or glucosaminoglycans), proteins (for example, collagen or fibroin), including recombinant proteins, or combinations of the referred macromolecules. Cross-linking agents can also be added during or after the production of the membrane.

The selected polymer is solubilized in an adequate solvent, including aqueous or organic solvents, or melted into a liquid phase.

The osteoconductive material is selected among natural or inorganic systems that may be processed in the shape of particles or granules. Examples of such materials are calcium phosphates, such as hydroxyapatite or tricalcium phosphate, calcium deficient hydroxyapatite, calcium phosphates doped with other elements, Bioglass® and other bioactive glasses and glass-ceramics, calcium sulfate, or combinations among such systems. Examples of natural-origin materials are bone particles, or materials containing calcium derived from corals or mineralized algae. It is possible to use particles with different crystallinity degrees or different phase compositions, that may be obtained from appropriate thermal treatments. The process is compatible with the use of particles featuring different geometries and shapes, which could be regular or irregular, isotropic or anisotropic, including geometries such as plates or fibers. The dimension of such objects may vary between 5 nanometers up to 2 millimeters, more desirably in the range of about 50 nanometers to about 500 micrometers.

The surface of the particles may be modified with the objective of improving the interaction with the polymeric component. Different chemical methods may be used in this context, in order to control the surface chemistry, the surface electrical charge, the wettability or to graft the same (or a compatible) polymer that will be used to produce the membrane, using, for example, surface initiated polymerization reaction.

The polymer is initially solubilized in a liquid solvent, organic or not, in concentrations between about 0.1% and about 20% of weight of polymer/volume of solvent, more desirably in most instances in the range of about 0.5% w/v to about 5% w/v. The polymer may be also found in a liquid form by the action of temperature. The particles are suspended in the liquid phase containing the polymer, desirably under agitation, in concentrations that can vary between 5% and 60% of weight of particles/volume of liquid. The concentration of the materials and the quantity of the final suspension will determine the thickness or mass of the membrane. The suspension should ideally be homogeneous at the beginning to guarantee that the final membrane exhibits a constant concentration of osteoconductive material in the entire surface.

It is also possible to include other substances in the formulation composed by polymeric and osteoconductive constituents, including salts, pH adjusters, dispersing agents, tensioactive and thickening agents, that could control the dispersion of the particles and the concentration gradient of the particles along the thickness of the final membrane.

Other therapeutic molecules can also be incorporated in the membrane, including drugs (for example, antibiotics or anti-inflammatories), growth and differentiation agents, or other therapeutic proteins. Such molecules can be dispersed directly in the liquid formulation or previously encapsulated into nano- or microparticles. Such pre-encapsulation also allows the establishment of concentration gradients of such particles during the fabrication of the membrane, extending the concept of asymmetric properties of such device: besides having a membrane with asymmetric osteoconductive properties, the same device could deliver therapeutic molecules also in an asymmetric form. The molecules could be also introduced after the processing of the membrane, by immersing it in a solution containing the therapeutic molecules in the free form or previously encapsulated in nano/microparticles.

During the elimination of the solvent, or solidification of the membrane, the particles are deposited towards one of the face of the membrane. This process may be induced by gravity, but also by the action of other fields, such as electric or magnetic, if the particles react to the action of such fields. The gradient in the concentration of the particles is the result of the combination of the rate of the solidification of the membrane (mainly dependent, for example, on the rate of solvent evaporation) and the speed of deposition of the osteoconductive towards one of the face of the membrane. The density of the particles should be between 0.8 and 6, and should be ideally higher than the density of the suspension. The suspension should be ideally placed in a chamber with controlled temperature and pressure, in which the extraction of the vapor resulting from solvent evaporation can be performed. The rate of solvent evaporation may be tuned through the increase of temperature or decrease of pressure.

The obtained flexible membrane is then dried, using, for example, a vacuum oven.

The thickness of the membrane could vary between 20 micrometers and 3 millimeters, more desirably in most instances in the range of about 200 micrometers to about 1000 micrometers. The methodology presented allows the production of a membrane with a continuous change or asymmetric concentration of particles along the thickness of the membrane, in which one step of solvent elimination may be sufficient.

The bioactive particles do not necessarily need to be exposed in the surface of the membrane. They can be involved by the polymer, but the concentration of particles in one of the face of the membrane should be high enough so that the device exhibits osteoconductive properties in that face. This process is facilitated if the polymer processes water uptake ability.

The invention permits the production of an homogeneous membrane with an area higher than 20×20 cm2. The shape of the base onto which the membrane is produce will dictate its geometry. A flat membrane can be obtained using a plain mould. It is then possible to obtain membranes with more complex shapes, such as convex and concave.

The two surfaces of the membrane may be modified independently or simultaneously, using physicochemical methods, including the combination of different methods. Parameters could be changed, including wettability, surface energy or roughness. Biomolecules, such as peptides, proteins or antibodies, could be also grafted or adsorbed onto one or the two faces of the membrane, in order to control the biological function of surrounded cells and tissues. The modifications can also be performed in all the volume of the membrane, during or after its preparation; for example, the cross-linking of the polymeric phase may be achieved using appropriate agents, in order to control the mechanical properties, the swelling capability or the permeability of the membrane.

The obtained membrane can be cut with the desired contour and size that are adequate for the specific application.

Besides being possible to control the contour of the membrane, hollows may be produced in the membrane so that the implant that is coated with the membrane may be partially in contact with the surrounded tissues.

After the production, the membrane may be sterilized and packed. The membrane may be sterilized, for example, with gamma radiation or ethylene oxide. Most of the operations should be carried out in sterile conditions, using, for example, a clean room.

During the application, the membrane may be combined with other membranes (synthetic or biological), obtained by stacking the different elements.

The membranes may be fixed, using, for example, sutures or surgical adhesives (e.g. fibrin glue). The membrane may be also placed in the site without the need of any direct fixation, using, for example, another biological or synthetic membrane that is fixed over the membrane.

The membrane may be produced using polymers with water uptake capability, such as, using chitosan-based materials. In such cases, the membranes are impermeable to cells but allow the diffusion of nutrients for cells and fluid transport.

The membrane produced according to this invention can be used in medicine or veterinary medicine in the following forms:
As a material to guide the regeneration of mineralized tissues, in which the cellular growth is promoted in one of the face of the composite membrane, and the ensemble prevents the growth and migration of undesired cells, as well as, offering a mechanical barrier resulting from the pressure difference existing between both faces of the membrane. The side of the membrane featuring higher concentration of osteoconductive elements is faced to the region of the defect or the region in which it is intended the conduction of mineralized tissue. Upon implantation it is expected a better osteointegration of the membrane in that face. During the degradation of the membrane the concentration of the osteoconductive elements in contact with the surrounded mineralized tissue will expectedly decrease due to solubilization or leaching. This process accompanies the progressively decrease in the need of apatite deposition, during the progress of osteointegration and development of de novo mineralized tissue. The other face of the membrane constitutes a barrier impermeable to undesirable cells. The membrane permits also the release of therapeutic molecules to the surrounded tissues.
as a sealant, to fix cells or osteoconductive material incorporated in the bone or maxilofacial defect.
as a sealant of a tissue engineering device, including porous structures, granular materials or hydrogels, with our without cells.
as a membrane to envelope the surface of implants with a low osteoconductivity, to be used in orthopedic or odontological applications. In this particular case the non-oesteoconductive face could have adhesive properties or may be glued to the surface of the implant. The membrane can also be sutured after enveloping the implant. The membrane should be flexible so that the surface of non-flat implants could be effectively covered.

The uses of the terms “a” and “an” and “the” and similar references in the context of describing the invention (especially in the description of the claims) are to be interpreted to cover both singular and plural, unless otherwise indicated herein or clearly contradicted by context. Recitation of ranges of values herein are merely intended to serve as a shorthand method of referring individually to each separate value falling within the range, unless otherwise indicated herein, and each separated value is incorporated into specification as if it were individually recited herein. All methods described herein can be performed in any suitable order unless otherwise stated or otherwise clearly contradicted by context. The use of any and all examples, or exemplary language (e.g. “such as”, “including”, “for example”) provided herein, is intended merely to better illuminate the invention and does not pose a limitation on the scope of the invention unless otherwise claimed. No language in the specification should be construed as indicating any non-claimed element as essential to the practice of the invention.

In this patent, the terms “osteoconductive” and “osteoconductivity” could be, in most of the places, replaced by the terms “osteoinductive” and “osteoinductivity”, respectively. In the context of this patent, one could be envisage to produce membranes with asymmetric osteoinductive properties, for the same kind of applications. In this case, the membranes featuring different concentration of osteoinductive materials along the thickness would be prepared.

### Example

The proof-of-concept of this invention was done by producing a flexible membrane, with about 0.2 mm and exhibiting osteoconductive properties just in one of the face, through the formation of a gradient in concentration or asymmetric disposition of osteoconductive particles. This membrane aimed at being used in guided bone regeneration. Therefore it should be flexible enough to be adapted to possible non-flat contours of the site of implantation. The membrane should be thick enough and have sufficient strength to warrant the mechanical and geometrical integrity during its utilization, as well as to warrant the possibility of being sutured. Materials already approved to be implanted in humans were used in this example.

In this case a biodegradable polyester material, based on poly(lactic acid) was used: poly(D,L-lactic acid), PDLLA. The validation of this process using PDLLA could be extensive to other biodegradable aliphatic polyesters.

For the osteoconductive materials, 45S5 Bioglass® was used (BG). BG has the following composition (in weight %): 45.0 SiO2, 24.5 CaO, 24.5 Na2O, 6.0 P2O5. BG has been widely used in different orthopedic applications and is approved by FDA to be used in humans.

The membrane should be able to prevent the deposition of a film of calcium phosphate in one of the faces until, at least, 14 days, in an in vitro bioactivity test. The other face should promote the deposition of apatite, ideally after 5 days of immersion in a fluid with a similar ionic composition of human blood plasma.

### Preparation of the Membrane and Characterization

40 g of PDLLA, with molecular weight Mn=3170 and Mw=100000, and 10 g of BG particles, with sizes lower than 20 micrometers are dissolved and dispersed in 30 g of chloroform. The mixture is casted in a Petri dish, such that a membrane with uniform thickness could be formed, upon solvent evaporation. The membrane is dried in a vacuum oven at 40° C., during 48 hours, and detached from the Petri dish.

The obtained membrane has two faces exhibiting an asymmetry in the concentration of BG particles. To confirm such feature, in vitro bioactivity tests were performed. The membrane was immersed during different time periods in “simulated body fluid”, SBF, a solution with an ionic composition similar to that found in human blood plasma. The ionic compositions (in mM/dm3) of the used SBF and of the blood plasma are the following:

| ion | SBF | Blood plasma |
|---|---|---|
| Na+ | 142.0 | 142.0 |
| K+ | 5.0 | 5.0 |
| Mg2+ | 1.5 | 1.5 |
| Ca2+ | 2.5 | 2.5 |
| Cl− | 147.8 | 103.0 |
| HCO3− | 4.2 | 27.0 |
| HPO4 3− | 1.0 | 1.0 |
| SO4 2− | 0.5 | 0.5 |

After immersion of the membrane in SBF, the two faces were analyzed. Such methodology allows to evaluate the bioactive character of the surface of a material, that could be assessed by the speed and magnitude of the precipitation of carbonated hydroxyapatite (HA) onto the surface of the material; such calcification ability could be correlated with the osteoconductive character of the material in vivo (e.g. T. Kokubo and H. Takadama, Biomaterials 27 (2006) 2907).

For the case of the membrane produced in this example, the immersion times were 1, 3, 7, 14 and 21 days. The two sides of the membrane were analyzed by scanning electronic microscopy (SEM), and the nature of the inorganic film that precipitate was analyzed by infrared spectroscopy (FTIR) and by X-ray diffraction (XRD).

### Results

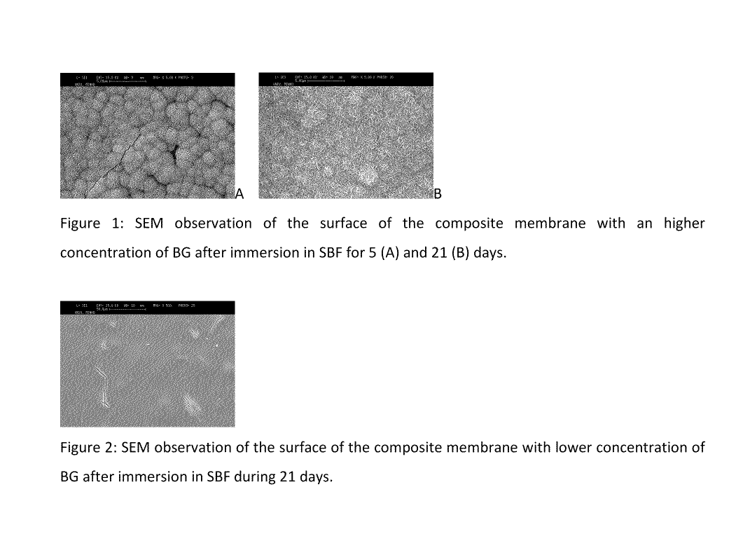

FIG. 1 presents the micrographs of the more osteoconductive face of the membrane after 5 and 21 days of immersion in SBF. It may be seen that in the two cases a ceramic layer was formed on the surface; the morphology of this calcified layer is consistent with the precipitates deposited in bioactive materials after immersion in SBF, in which a HA layer is formed exhibiting a cauliflower texture, constituted by the agglomeration of needle-like HA crystals. No calcification took place in the other side of the membrane, even after 21 days immersion in SBF (see FIG. 2).

The precipitate formed was scraped from the surface such as the one showed in FIG. 1 and analyzed by FTIR. Absorption bands were identified and assigned to the existence of phosphate and carbonated groups, being consistent with the presence of carbonated HA. XRD results showed the presence of diffraction peaks compatible with HA. Moreover, such peaks have low intensity and are large, similar to the spectrum observed in HA formed in bone.

### CONCLUSIONS

It can be concluded that the membrane produced in this example features a clear asymmetric bioactivity: after immersion in SBF an homogeneous layer of HA is formed in the more osteoconductive face, where the concentration of BG in the membrane is higher; in the other face, where the content of BG is lower, no calcification takes place, even after 21 days of immersion in SBF.

1. A bioresorbable and flexible membrane for applications in regenerative medicine and in reconstructive surgery, constituted by, at least: (1−) a biodegradable macromolecular fraction and (2−) an osteoconductive material with the shape of particles or granules, exhibiting a gradual variation, or an asymmetry, of concentration of the said osteoconductive material along the thickness of the membrane, so that the said membrane may exhibit higher osteoconductive properties in one of the faces.

2. A membrane as claimed in claim 1, wherein said osteoconductive material is confined inside the said membrane, not being exposed in the surface.

3. A process to produce the membrane as claimed in claim 1 with a thickness between about 20 micrometers and about 2 millimeters.

4. A membrane as claimed in claim 1, containing the biodegradable macromolecular fraction, selected among synthetic and natural-based polymers, more preferable among polyesters or polyssacharides.

5. A membrane as claimed in claim 1, wherein said osteoconductive material is based on ceramic or glass particles, with a synthetic or natural origin.

6. A production process of said membranes claimed in claim 1 that includes the following steps:
combination of the macromolecular fraction and the osteoconductive material with a solvent, in which the polymer is dissolved or dispersed and the particles of osteoconductive material are dispersed;
solvent extraction;
gradual deposition of the particles towards one of the face of the film formed;
chemical modification.

7. A process of membrane production as claimed in claim 6 wherein the chemical modification step is not considered.

8. A process of membrane production as claimed in claim 6, wherein solvent extraction takes place in the solid or liquid phase.

9. A process of membrane production as claimed in claim 6, wherein solvent extraction takes place by the action of temperature or pressure, or by the addition of a non-solvent.

10. A process of membrane production as claimed in claim 6, wherein said particles deposition is induced by the action of a gravitational, electric or magnetic field.

11. A process of membrane production as claimed in claim 6 wherein said chemical modification takes place during or after solvent removal.

12. A process of membrane production as claimed in claim 6 wherein said chemical modification is performed in either one or both faces of the membrane, or in the all polymer fraction.

13. A process of membrane production as claimed in claim 6 wherein said chemical modification may be: a cross-linking process of the polymeric fraction; a method to modify the acid-base character of the polymer; a method to change the wettability of the membrane; a process to graft or immobilize biomolecules, including peptides or proteins.

14. A process of membrane production as claimed in claim 6, wherein therapeutic molecules are introduced.

15. A process of membrane production as claimed in claim 14 wherein said therapeutic molecules are introduced on the initial formulation or after membrane processing, through the immersion of the membrane in a solution containing the therapeutic molecules.

16. A process of membrane production as claimed in claim 14, wherein said therapeutic molecules are introduced in a free form.

17. A process of membrane production as claimed in claim 14, wherein said therapeutic molecules are previously encapsulated in nano or microparticles before being introduced in the membrane.

18. The use of a membrane as claimed in claim 1, wherein said membrane is placed or fixed in an anatomical defect, in which the osteoconductive side of the membrane is facing the region where it is required the regeneration, protection or reconstruction of mineralized tissue.

19. The use of a membrane as claimed in claim 18, wherein said anatomical defect is previously filled with synthetic or biological material that enable the regeneration of the tissue.

20. The use of a membrane as claimed in claim 19, wherein said material is a porous structure, particles or an hydrogel.

21. The use of a membrane as claimed in claim 18, wherein said filling material is combined with cells or growth factors, consistent with tissue engineering or regenerative medicine strategies.

22. The use of a membrane as claimed in claim 1, to cover partially or totally the surface of an implantable device for orthopedic or odontological applications.

23. The use of a membrane as claimed in claim 18, wherein said material is applied in reconstructive maxillofacial surgery.
